(19) United States
(12) Patent Application Publication
(10) Pub. No.: US 2024/0081671 A1
(43) Pub. Date: Mar. 14, 2024

(54) WIRELESS TECHNOLOGIES FOR HEALTH MONITORING

(71) Applicants: Walid El Hajj, Antibes (FR); Elad Shababo, Givat Ada (IL)

(72) Inventors: Walid El Hajj, Antibes (FR); Elad Shababo, Givat Ada (IL)

(21) Appl. No.: 17/943,394

(22) Filed: Sep. 13, 2022

(51) Int. Cl. A61B 5/05; A61B 5/00; A61B 5/145; G08B 21/18

(52) U.S. Cl. CPC A61B 5/05; A61B 5/14532; A61B 5/6898; G08B 21/18

(57) ABSTRACT

An apparatus can include an antenna, transmitter circuitry coupled to the antenna, and processing circuitry coupled to the transmitter circuitry. The processing circuitry can measure a first reflection coefficient of the antenna in free space and a second reflection coefficient of the antenna when the antenna is proximate biological tissues. The processing circuitry can determine a contextual property of the biological tissues based on a comparison between the first coefficient and the reflection coefficient proximate biological tissues.

### TECHNICAL FIELD

Aspects of the disclosure pertain to health monitoring. More particularly, aspects relate to wireless technologies for detecting blood substance concentration or other tissues characteristics.

### BACKGROUND

Many people require specific health monitoring. However, access to health monitoring and other services may be high in cost and cumbersome to achieve. In particular, some health monitoring such as blood sugar measurement is invasive and not easily accessible to all population or in low-income countries. This leads to a higher health risk and cost as well as unhealthy lifestyles. Therefore, there is a general need to provide more convenient monitoring.

### BRIEF DESCRIPTION OF THE DRAWINGS

In the drawings, which are not necessarily drawn to scale, like numerals may describe similar components in different views. Like numerals having different letter suffixes may represent different instances of similar components. Some aspects are illustrated by way of example, and not limitation, in the figures of the accompanying drawings in which:

FIG. 1A illustrates an exemplary user device for health monitoring according to some aspects.

FIG. 2A illustrates exemplary wireless communication circuitry according to some aspects.

FIG. 2B illustrates aspects of exemplary transmit circuitry illustrated in FIG. 2A according to some aspects.

FIG. 2C illustrates aspects of exemplary transmit circuitry illustrated in FIG. 2A according to some aspects.

FIG. 2D illustrates aspects of exemplary radio frequency circuitry illustrated in FIG. 2A according to some aspects.

FIG. 2E illustrates aspects of exemplary receive circuitry in FIG. 2A according to some aspects.

FIG. 3 illustrates a method of use of the apparatus according to some aspects of the disclosure.

FIG. 4A illustrates an example antenna platform for glucose measurement.

FIG. 4B illustrates electromagnetic field strength during glucose measurement.

FIG. 5 illustrates VSWR variation according to frequency.

FIG. 6 illustrates the difference in antenna reflection coefficient between free space and plasma at different permittivities.

FIG. 7 illustrates a relationship between antenna reflection coefficient variation and plasma permittivity.

FIG. 8 illustrates a relationship between permittivity and glucose level.

FIG. 9 illustrates a relationship between antenna reflection coefficient variation and glucose level.

FIG. 10 illustrates a method for blood substance concentration measurement in accordance with some aspects.

FIG. 11 illustrates a block diagram of a communication device in accordance with some aspects.

FIG. 12 illustrates a system level diagram, depicting an example of an electronic device in accordance with some aspects.

### DETAILED DESCRIPTION

The following description and the drawings sufficiently illustrate specific aspects to enable those skilled in the art to practice them. Other aspects may incorporate structural, logical, electrical, process, and other changes. Portions and features of some aspects may be included in, or substituted for, those of other aspects. Aspects set forth in the claims encompass all available equivalents of those claims.

In modern society, many people spend hours per day using laptops and other computing devices. At the same time, many people require specific health monitoring, while others are interested in wellness services like nutrition, stress management, fitness services. However, the access to this monitoring and these services can be high in cost, invasive, or inconvenient. For example, diabetics requiring blood sugar monitoring are often inconvenienced by invasive blood sugar measurement instruments. This can lead to a higher health risk and health care costs for the patients themselves as well as for the rest of society.

To address these concerns, some aspects of this disclosure use wireless technologies, on available devices that users already spend a lot of time using, to monitor specific health metrics when the patient or person is within proximity of that device.

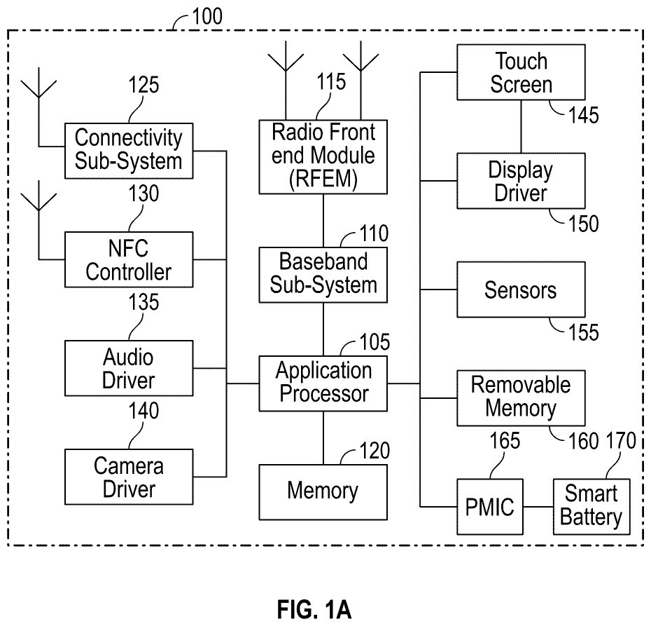

FIG. 1A illustrates an exemplary user device for health monitoring according to some aspects. The user device 100 may be a mobile device or a laptop device in some aspects and includes an application processor 105, baseband processor 110 (also referred to as a baseband sub-system), radio front end module (RFEM) 115, memory 120, connectivity sub-system 125, near field communication (NFC) controller 130, audio driver 135, camera driver 140, touch screen 145, display driver 150, sensors 155, removable memory 160, power management integrated circuit (PMIC) 165, and smart battery 170.

In some aspects, application processor 105 may include, for example, one or more central processing unit (CPU) cores and one or more of cache memory, low drop-out voltage regulators (LDOs), interrupt controllers, serial interfaces such as SPI, I2C or universal programmable serial interface sub-system, real time clock (RTC), timer-counters including interval and watchdog timers, general purpose IO, memory card controllers such as SD/MMC or similar, USB interfaces, MIPI interfaces, and/or Joint Test Access Group (JTAG) test access ports.

In some aspects, baseband processor 110 may be implemented, for example, as a solder-down substrate including one or more integrated circuits, a single packaged integrated circuit soldered to a main circuit board, and/or a multi-chip module including two or more integrated circuits.

Applications of mmWave technology can include, for example, WiGig and future 5G, but the mmWave technology can be applicable to a variety of telecommunications systems. The mmWave technology can be especially attractive for short-range telecommunications systems. WiGig devices operate in the unlicensed 60 GHz band, whereas 5G mmWave is expected to operate initially in the licensed 28 GHz and 39 GHz bands.

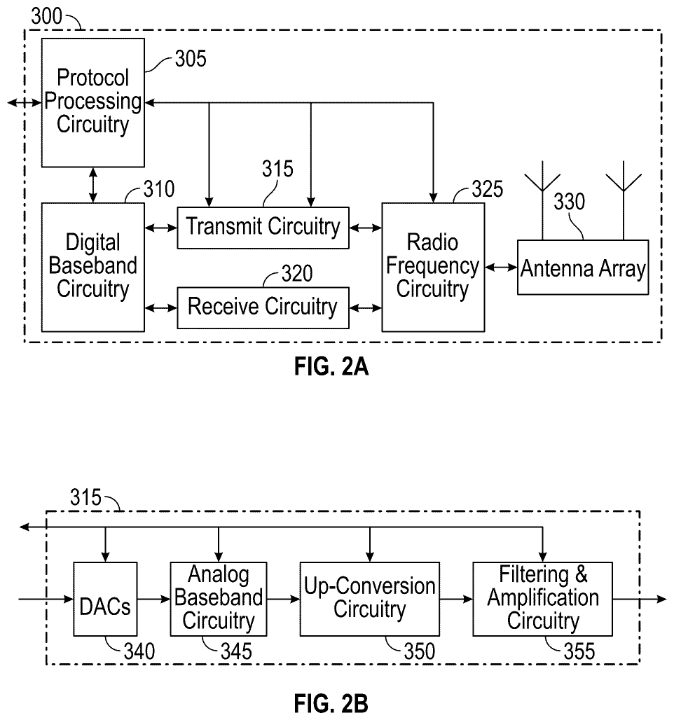

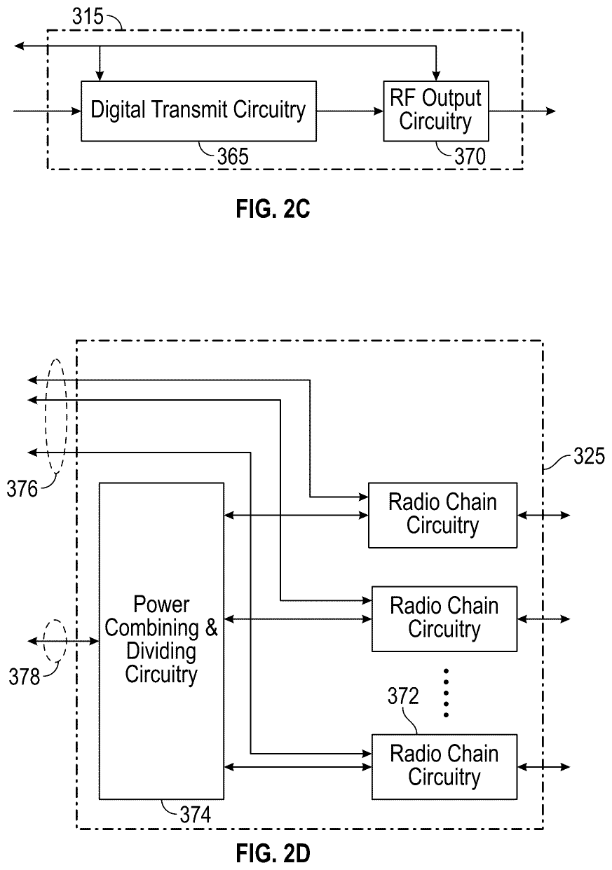

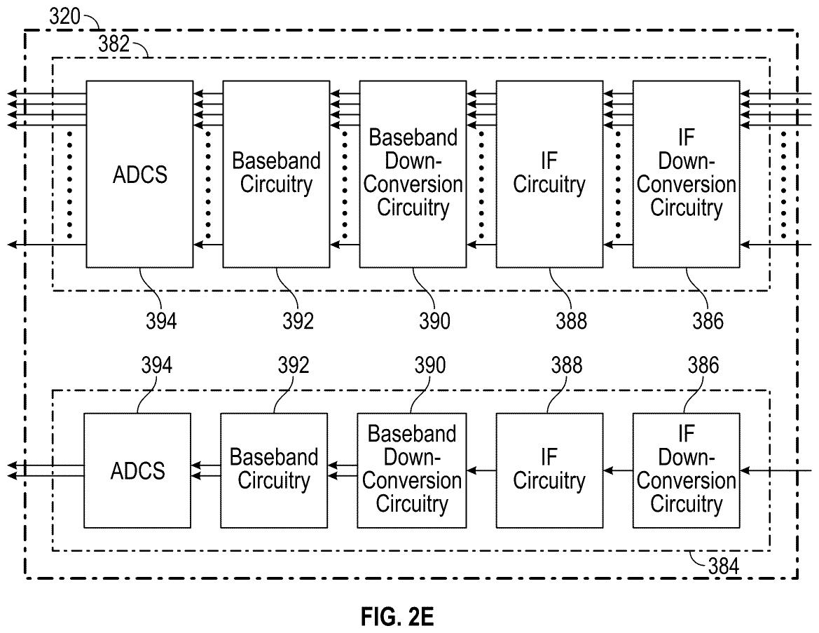

FIG. 2A illustrates exemplary wireless communication circuitry according to some aspects; FIGS. 2B and 2C illustrate aspects of transmit circuitry shown in FIG. 2A according to some aspects; FIG. 2D illustrates aspects of radio frequency circuitry shown in FIG. 2A according to some aspects; FIG. 2E illustrates aspects of receive circuitry in FIG. 2A according to some aspects. Wireless communication circuitry 300 shown in FIG. 2A may be alternatively grouped according to functions. Components illustrated in FIG. 2A are provided here for illustrative purposes and may include other components not shown in FIG. 2A.

Wireless communication circuitry 300 may include protocol processing circuitry 305 (or processor) or other means for processing. Protocol processing circuitry 305 may implement one or more of medium access control (MAC), radio link control (RLC), packet data convergence protocol (PDCP), radio resource control (RRC) and non-access stratum (NAS) functions, among others. Protocol processing circuitry 305 may include one or more processing cores to execute instructions and one or more memory structures to store program and data information.

Wireless communication circuitry 300 may further include digital baseband circuitry 310. Digital baseband circuitry 310 may implement physical layer (PHY) functions including one or more of hybrid automatic repeat request (HARQ) functions, scrambling and/or descrambling, coding and/or decoding, layer mapping and/or de-mapping, modulation symbol mapping, received symbol and/or bit metric determination, multi-antenna port pre-coding and/or decoding which may include one or more of space-time, space-frequency or spatial coding, reference signal generation and/or detection, preamble sequence generation and/or decoding, synchronization sequence generation and/or detection, control channel signal blind decoding, and other related functions.

Wireless communication circuitry 300 may further include transmit circuitry 315, receive circuitry 320 and/or antenna array circuitry 330. Wireless communication circuitry 300 may further include RF circuitry 325. In some aspects, RF circuitry 325 may include one or multiple parallel RF chains for transmission and/or reception. Each of the RF chains may be connected to one or more antennas of antenna array circuitry 330.

In some aspects, protocol processing circuitry 305 may include one or more instances of control circuitry. The control circuitry may provide control functions for one or more of digital baseband circuitry 310, transmit circuitry 315, receive circuitry 320, and/or RF circuitry 325.

FIGS. 2B and 2C illustrate aspects of transmit circuitry shown in FIG. 2A according to some aspects. Transmit circuitry 315 shown in FIG. 2B may include one or more of digital to analog converters (DACs) 340, analog baseband circuitry 345, up-conversion circuitry 350 and/or filtering and amplification circuitry 355. DACs 340 may convert digital signals into analog signals. Analog baseband circuitry 345 may perform multiple functions as indicated below. Up-conversion circuitry 350 may up-convert baseband signals from analog baseband circuitry 345 to RF frequencies (e.g., mmWave frequencies). Filtering and amplification circuitry 355 may filter and amplify analog signals. Control signals may be supplied between protocol processing circuitry 305 and one or more of DACs 340, analog baseband circuitry 345, up-conversion circuitry 350 and/or filtering and amplification circuitry 355.

Transmit circuitry 315 shown in FIG. 2C may include digital transmit circuitry 365 and RF circuitry 370. In some aspects, signals from filtering and amplification circuitry 355 may be provided to digital transmit circuitry 365. As above, control signals may be supplied between protocol processing circuitry 305 and one or more of digital transmit circuitry 365 and RF circuitry 370.

FIG. 2D illustrates aspects of radio frequency circuitry shown in FIG. 2A according to some aspects. Radio frequency circuitry 325 may include one or more instances of radio chain circuitry 372, which in some aspects may include one or more filters, power amplifiers, low noise amplifiers, programmable phase shifters and power supplies.

Radio frequency circuitry 325 may also in some aspects include power combining and dividing circuitry 374. In some aspects, power combining and dividing circuitry 374 may operate bidirectionally, such that the same physical circuitry may be configured to operate as a power divider when the device is transmitting, and as a power combiner when the device is receiving. In some aspects, power combining and dividing circuitry 374 may include one or more wholly or partially separate circuitries to perform power dividing when the device is transmitting and power combining when the device is receiving. In some aspects, power combining and dividing circuitry 374 may include passive circuitry including one or more two-way power divider/combiners arranged in a tree. In some aspects, power combining and dividing circuitry 374 may include active circuitry including amplifier circuits.

In some aspects, radio frequency circuitry 325 may connect to transmit circuitry 315 and receive circuitry 320 in FIG. 2A. Radio frequency circuitry 325 may connect to transmit circuitry 315 and receive circuitry 320 via one or more radio chain interfaces 376 and/or a combined radio chain interface 378. In some aspects, one or more radio chain interfaces 376 may provide one or more interfaces to one or more receive or transmit signals, each associated with a single antenna structure. In some aspects, the combined radio chain interface 378 may provide a single interface to one or more receive or transmit signals, each associated with a group of antenna structures.

FIG. 2E illustrates aspects of receive circuitry in FIG. 2A according to some aspects. Receive circuitry 320 may include one or more of parallel receive circuitry 382 and/or one or more of combined receive circuitry 384. In some aspects, the one or more parallel receive circuitry 382 and one or more combined receive circuitry 384 may include one or more Intermediate Frequency (IF) down-conversion circuitry 386, IF processing circuitry 388, baseband down-conversion circuitry 390, baseband processing circuitry 392 and analog-to-digital converter (ADC) circuitry 394. As used herein, the term “intermediate frequency” refers to a frequency to which a carrier frequency (or a frequency signal) is shifted as in intermediate step in transmission, reception, and/or signal processing. IF down-conversion circuitry 386 may convert received RF signals to IF. IF processing circuitry 388 may process the IF signals, e.g., via filtering and amplification. Baseband down-conversion circuitry 390 may convert the signals from IF processing circuitry 388 to baseband. Baseband processing circuitry 392 may process the baseband signals, e.g., via filtering and amplification. ADC circuitry 394 may convert the processed analog baseband signals to digital signals.

### Apparatus and System for Wireless Health Monitoring

As mentioned earlier herein, the user systems described above can be used for health monitoring in some aspects of the disclosure. By providing health monitoring (e.g., blood glucose monitoring or other blood substance level monitoring, or any biological monitoring such as blood pressure, cellular level counts, heart rate, etc.) on standard user devices, user convenience can be enhanced, and invasiveness and expense can be reduced or eliminated.

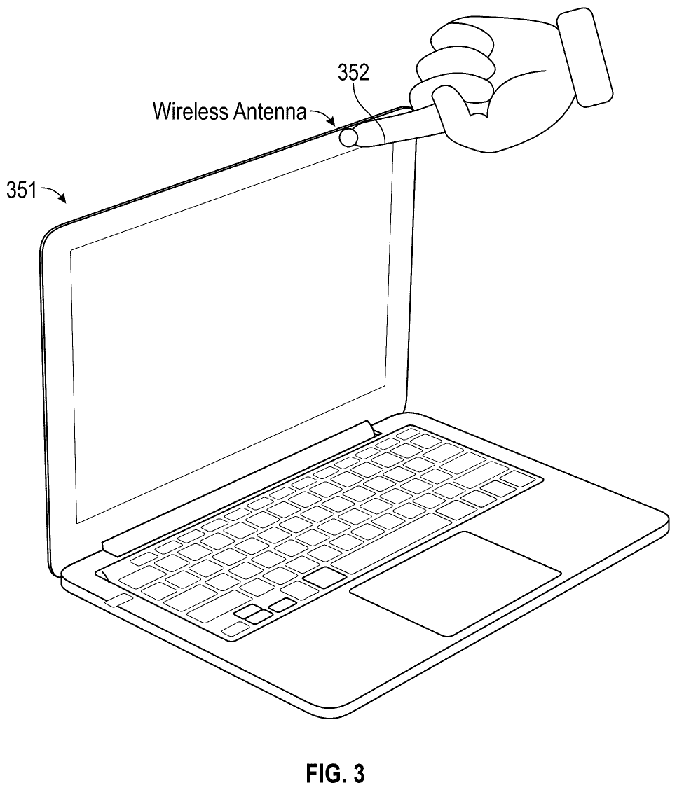

As such, an apparatus according to aspects can include an antenna (e.g., any of the antennas described above with reference to FIG. 1A-2) and transmitter circuitry coupled to the antenna. Processing circuitry (e.g., application processor 105 or other processing circuitry) can measure a free space reflection coefficient of the antenna in free space and another reflection coefficient of the antenna when the antenna is proximate biological tissues. The processing circuitry can determine a contextual property of the biological tissues based on a comparison between the free space reflection coefficient and the reflection coefficient proximate biological tissues. Further details regarding the reflection coefficients and antennas are provided below with reference to FIG. 3-9. The contextual property can be based on a context including user location, physiological attributes of the user (e.g., sex, age, weight, etc.), habits of the user (e.g., smoking, drug use, alcohol intake), and other attributes, actions or habits of the user.

FIG. 3 illustrates a method of use of the apparatus according to some aspects of the disclosure. When a user device, e.g., laptop 351 is booted or powered on, a wireless module of the laptop 351 (e.g., wireless components described with reference to the figures above) can be used to measure the antenna parameters and module settings in free space.

When presence of a user is detected, or when a user requests health monitoring, the user can be instructed or an indication can be provided for the user to place a body part (e.g., finger 352) in an area proximate a wireless antenna of the laptop 351. The wireless antenna can then measure the parameters variations between human presence and free space. For example, variations can be expressed in terms of antenna reflection coefficients.

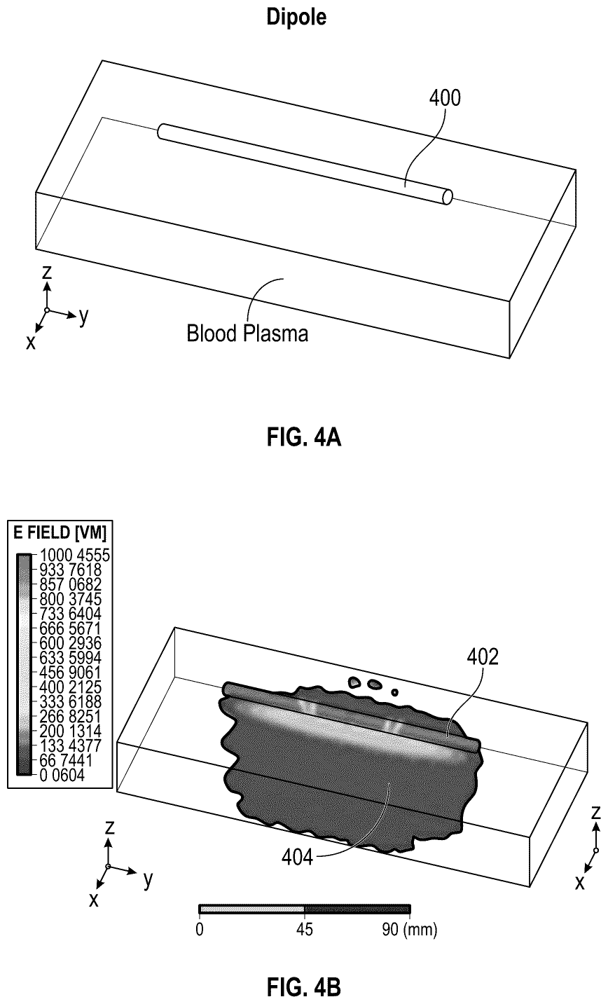

By way of example, an example antenna platform and simulated frequencies are provided in FIGS. 4A and 4B. In the illustrated an example, an antenna (e.g., dipole antenna) 400 can be brought proximate (e.g., about two millimeters) from a user's blood plasma. The distance can depend on varying factors such as antenna power parameters (S parameters) or parameters of the user device used. FIG. 4B illustrates electric field strength at a specific distance from the antenna. For example, at points 402, electromagnetic field strength is relatively strong, while at points 404 further from the antenna, the field strength weakens.

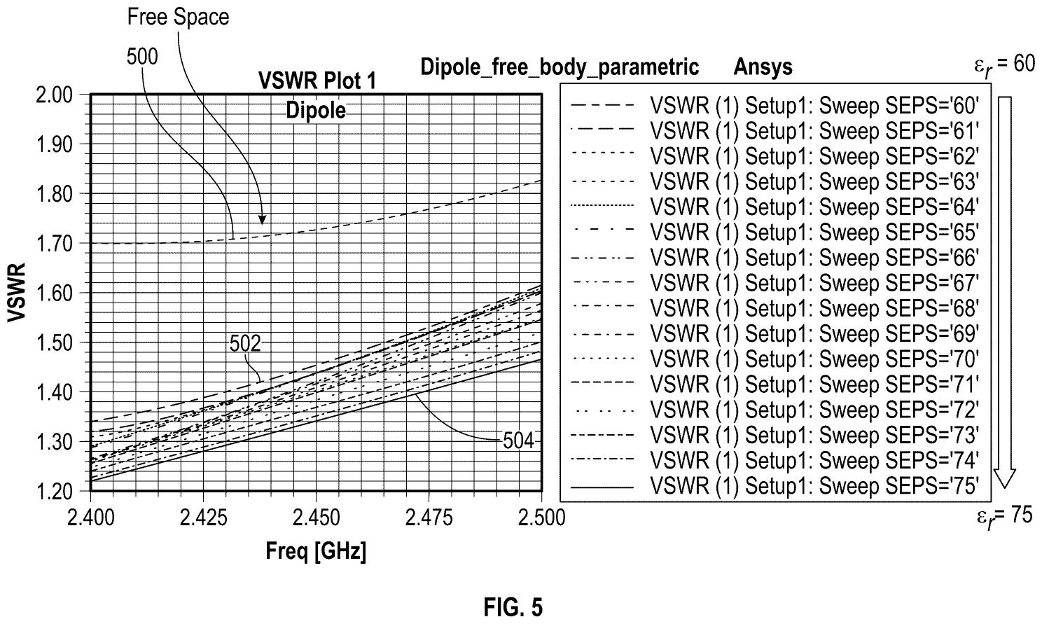

FIG. 5 illustrates how the antenna reflection coefficient of an antenna varies according to frequency. In simulations for determining human blood plasma permittivity values for different sugar (e.g., glucose) levels, blood plasma permittivity is changed from 60 to 75 to cover a range of sugar level between 0-16000 mg/dl for low band WiFi frequencies according to FIG. 5. Curve 500 illustrates the variation of the antenna reflection coefficient (ΔVSWR) in free space over various frequencies used in the 2.4 GHz band. It will be appreciated that similar curves and relationships can be obtained for frequencies in other commonly-used bands for user equipment, e.g., laptops and smart phones. For example, non-WiFi frequencies can be included, or acoustic measurements can be taken, or other frequency bands can be used, including user device frequency bands currently in use and for future use.

Similarly, curve 502 illustrates ΔVSWR in a liquid having permittivity of 60, and curve 504 illustrates ΔVSWR in a liquid having a permittivity of 75. The other curves illustrated are generated using values of between 61 and 74 permittivity.

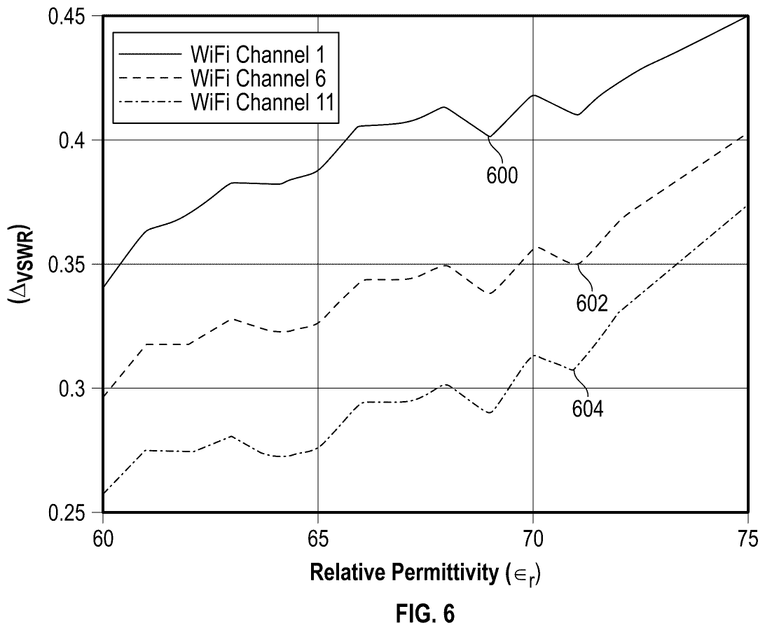

FIG. 6 illustrates the difference in antenna reflection coefficient between free space and plasma (ΔVSWR) at different permittivities. In examples, the permittivities can vary from 60-75. Curve 600 illustrates a relationship between ΔVSWR and permittivity for WiFi Channel 1. Curve 602 illustrates a relationship between ΔVSWR and permittivity for WiFi Channel 6. Curve 604 illustrates a relationship between ΔVSWR and permittivity for WiFi Channel 1.

Based on the results illustrated in FIG. 6, a mathematical relationship is established between ΔVSWR and plasma permittivity.

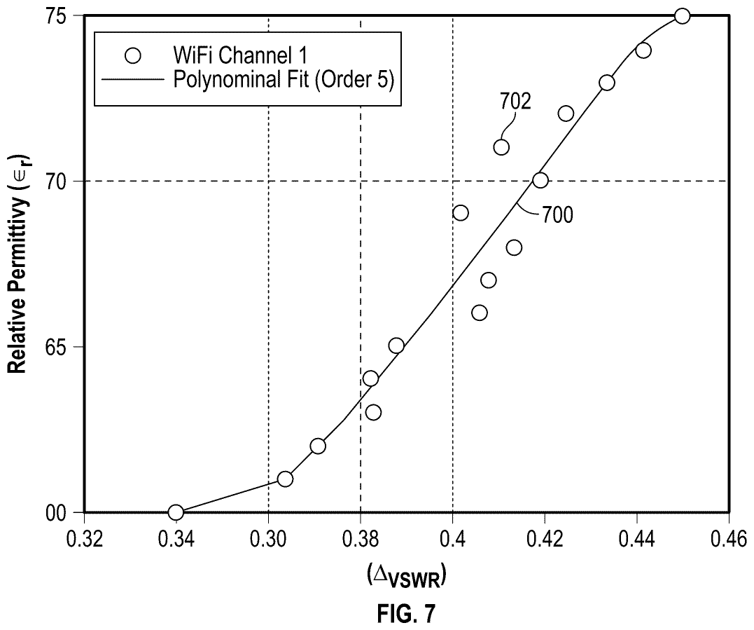

FIG. 7 illustrates a relationship 700 between ΔVSWR and plasma permittivity, where ΔVSWR is the difference between the antenna reflection coefficient in free space and in the presence of the finger. Data points 702 are captured by the processing circuitry based on measurements of the antenna reflection coefficient, and then curve fitting can be applied to generate relationship 700. The relationship between ΔVSWR and the plasma permittivity is based on known or advanced algorithms (curve fitting, artificial intelligence, machine learning, etc.) using data from simulation and/or measurement. In some examples, a lookup table or other stored data can be used or accessed to determine a relationship between the substance concentration level and (ΔVSWR). This table can or data can include data for multiple different frequencies and radio access technologies (RATs). For example, standard WiFi and cellular band frequencies can be tested so that relationships can be provided for multiple frequencies and bands used by the user device 351 (FIG. 3).

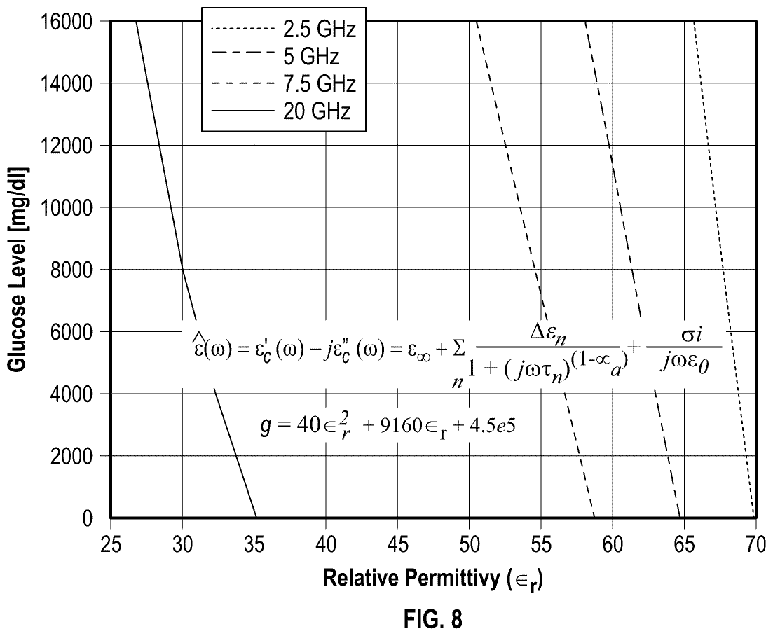

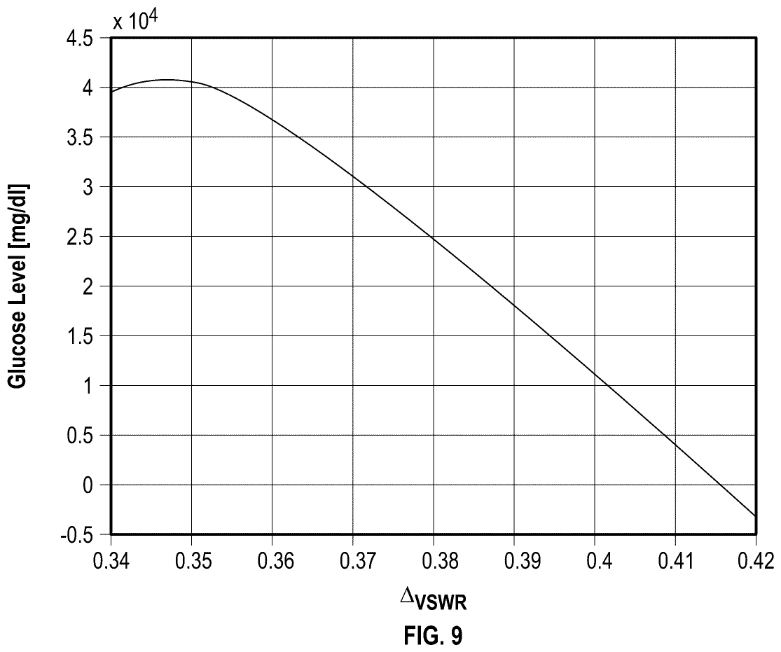

In turn, the relationship between blood plasma permittivity and the substance concentration can be used to determine glucose level according to known relationships between permittivity and glucose level as shown in FIG. 8. Therefore, a plot can be generated as in FIG. 9 to illustrate blood glucose level as a function of ΔVSWR. In summary, therefore, the variation in antenna reflection coefficient ΔVSWR can be converted to blood permittivity value corresponding to a specific concentration e.g., glucose.

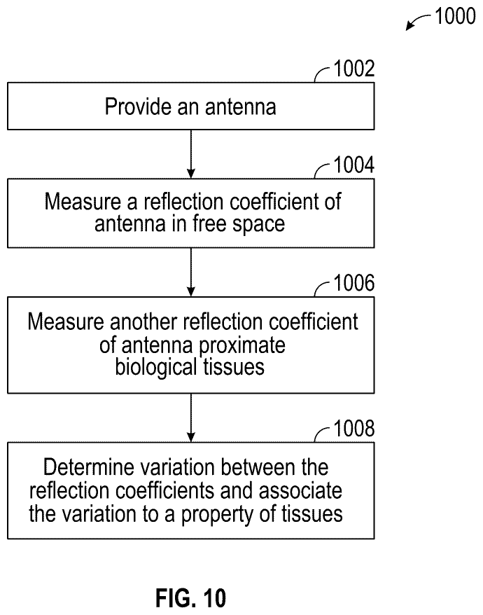

FIG. 10 illustrates a method 1000 for communicating in a wireless network according to some aspects. The method 1000 can be performed by components illustrated in the devices of FIGS. 1-3. The method 1000 can begin with operation 1002 by providing an antenna (or, for example, any antenna of an antenna array 330 (FIG. 2A).

The method 1000 can continue with operation 1004 with measuring a free space reflection coefficient of the antenna in free space.

The method 1000 can continue with operation 1008 with determining a contextual property of the biological tissues based on a comparison between the free space reflection coefficient and reflection coefficient proximate biological tissues. This contextual property can include glucose level and relationships for determining glucose level can be similar to those described in FIGS. 5-9 or other properties including blood pressure, heart rate, or concentrations of other substances besides glucose.

By performing method 1000 according to some aspects, blood substance concentration measurement can be accomplished using wireless technologies in devices already owned by a user, reducing user costs. Because antennas are used for measurement, invasiveness of blood draw procedures can be avoided.

### Other Systems and Apparatuses

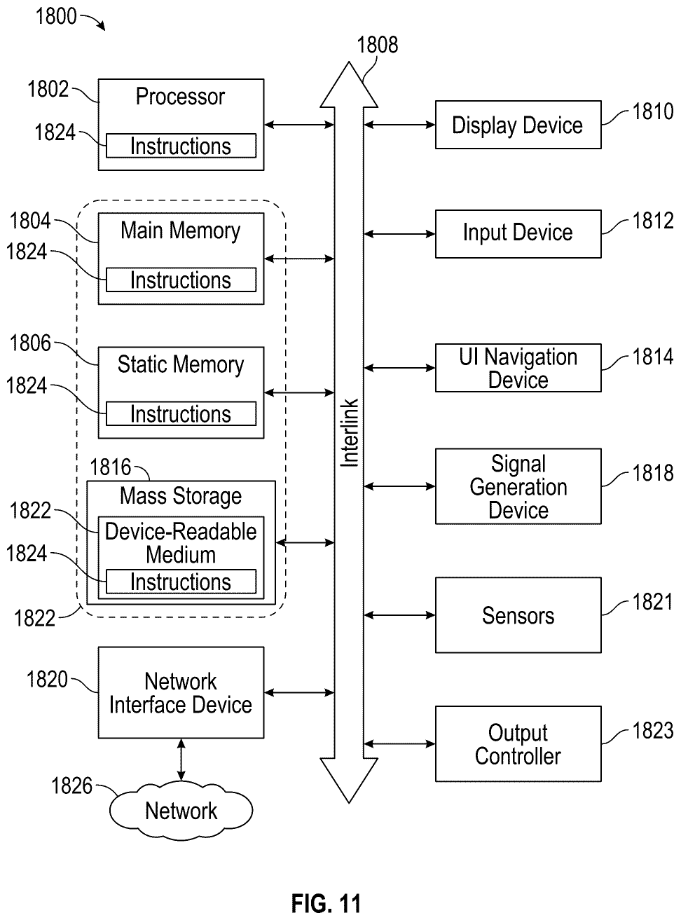

FIG. 11 illustrates a block diagram of a communication device 1800 such as an evolved Node-B (eNB), a new generation Node-B (gNB), an access point (AP), a wireless station (STA), a mobile station (MS), or a user equipment (UE), in accordance with some aspects. In alternative aspects, the communication device 1800 may operate as a standalone device or may be connected (e.g., networked) to other communication devices. In some aspects, the communication device 1800 can use one or more of the techniques and circuits discussed herein, in connection with any of FIG. 1A-FIG. 10.

Circuitry (e.g., processing circuitry) is a collection of circuits implemented in tangible entities of the device 1800 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation.

In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the device 1800 follow.

In some aspects, the device 1800 may operate as a standalone device or may be connected (e.g., networked) to other devices. In a networked deployment, the communication device 1800 may operate in the capacity of a server communication device, a client communication device, or both in server-client network environments. In an example, the communication device 1800 may act as a peer communication device in peer-to-peer (P2P) (or other distributed) network environment. The communication device 1800 may be a UE, eNB, PC, a tablet PC, a STB, a PDA, a mobile telephone, a smart phone, a web appliance, a network router, switch or bridge, or any communication device capable of executing instructions (sequential or otherwise) that specify actions to be taken by that communication device. Further, while only a single communication device is illustrated, the term “communication device” shall also be taken to include any collection of communication devices that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a communication device-readable medium. In an example, the software, when executed by the underlying hardware of the module, causes the hardware to perform the specified operations.

Accordingly, the term “module” is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

Communication device (e.g., UE) 1800 may include a hardware processor 1802 (e.g., a central processing unit (CPU), a graphics processing unit (GPU), a hardware processor core, or any combination thereof), a main memory 1804, a static memory 1806, and mass storage device 1816 (e.g., hard drive, tape drive, flash storage, or other block or storage devices), some or all of which may communicate with each other via an interlink (e.g., bus) 1808.

The communication device 1800 may further include a display unit 1810, an alphanumeric input device 1812 (e.g., a keyboard), and a user interface (UI) navigation device 1814 (e.g., a mouse). In an example, the display unit 1810, input device 1812 and UI navigation device 1814 may be a touch screen display. The communication device 1800 may additionally include a signal generation device 1818 (e.g., a speaker), a network interface device 1820, and one or more sensors 1821, such as a global positioning system (GPS) sensor, compass, accelerometer, or another sensor. The communication device 1800 may include an output controller 1823, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The mass storage device 1816 may include a communication device-readable medium 1822, on which is stored one or more sets of data structures or instructions 1824 (e.g., software) embodying or utilized by any one or more of the techniques or functions described herein. In some aspects, registers of the processor 1802, the main memory 1804, the static memory 1806, and/or the mass storage device 1816 may be, or include (completely or at least partially), the device-readable medium 1822, on which is stored the one or more sets of data structures or instructions 1824, embodying or utilized by any one or more of the techniques or functions described herein. In an example, one or any combination of the hardware processor 1802, the main memory 1804, the static memory 1806, or the mass storage device 1816 may constitute the device-readable medium 1822.

As used herein, the term “device-readable medium” is interchangeable with “computer-readable medium” or “machine-readable medium”. While the communication device-readable medium 1822 is illustrated as a single medium, the term “communication device-readable medium” may include a single medium or multiple media (e.g., a centralized or distributed database, and/or associated caches and servers) configured to store the one or more instructions 1824.

The term “communication device-readable medium” may include any medium that is capable of storing, encoding, or carrying instructions for execution by the communication device 1800 and that cause the communication device 1800 to perform any one or more of the techniques of the present disclosure, or that is capable of storing, encoding, or carrying data structures used by or associated with such instructions. Non-limiting communication device-readable medium examples may include solid-state memories, and optical and magnetic media. Specific examples of communication device-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); and CD-ROM and DVD-ROM disks. In some examples, communication device-readable media may include non-transitory communication device-readable media. In some examples, communication device-readable media may include communication device-readable media that is not a transitory propagating signal.

The instructions 1824 may further be transmitted or received over a communications network 1826 using a transmission medium via the network interface device 1820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, among others. In an example, the network interface device 1820 may include one or more physical jacks (e.g., Ethernet, coaxial, or phone jacks) or one or more antennas to connect to the communications network 1826. In an example, the network interface device 1820 may include a plurality of antennas to wirelessly communicate using at least one of single-input multiple-output (SIMO), MIMO, or multiple-input single-output (MISO) techniques. In some examples, the network interface device 1820 may wirelessly communicate using Multiple User MIMO techniques.

The term “transmission medium” shall be taken to include any intangible medium that is capable of storing, encoding, or carrying instructions for execution by the communication device 1800, and includes digital or analog communications signals or other intangible medium to facilitate communication of such software. In this regard, a transmission medium in the context of this disclosure is a device-readable medium.

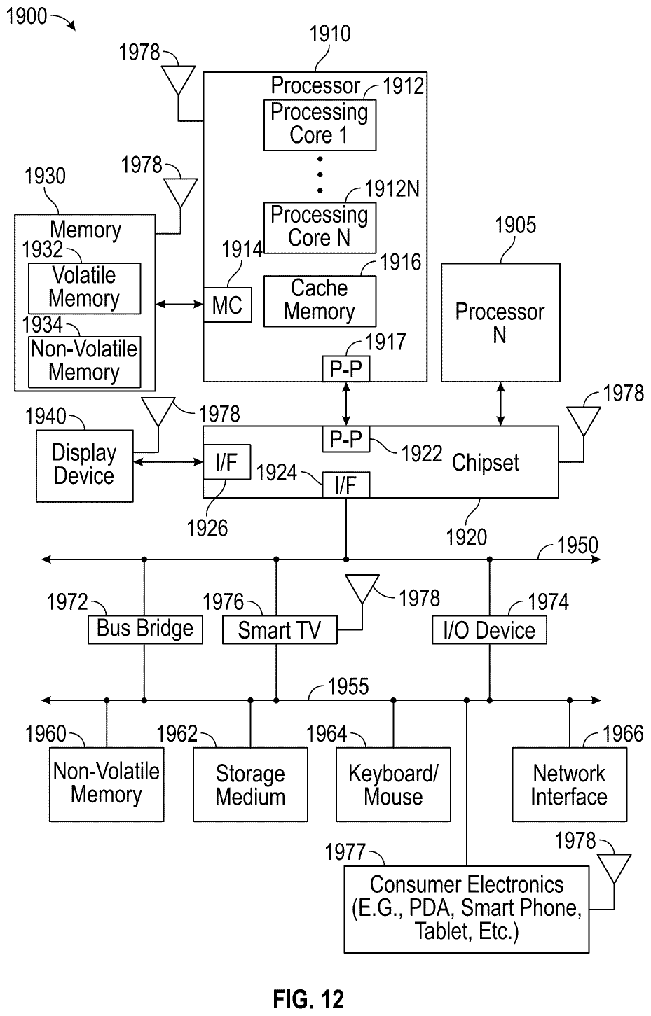

FIG. 12 illustrates a system level diagram, depicting an example of an electronic device (e.g., system) that can include, for example, a transmitter configured to selectively fan out a signal to one of multiple communication channels. FIG. 12 is included to show an example of a higher-level device application for the subject matter discussed above with regards to FIGS. 1-11. In one aspect, system 1900 includes, but is not limited to, a desktop computer, a laptop computer, a netbook, a tablet, a notebook computer, a personal digital assistant (PDA), a server, a workstation, a cellular telephone, a mobile computing device, a smart phone, an Internet appliance, or any other type of computing device. In some aspects, system 1900 is a system on a chip (SOC) system.

In one aspect, processor 1910 has one or more processor cores 1912, . . . , 1912N, where 1912N represents the Nth processor core inside processor 1910 where N is a positive integer. In one aspect, system 1900 includes multiple processors including 1910 and 1905, where processor 1905 has logic similar or identical to the logic of processor 1910. In some aspects, processing core 1912 includes, but is not limited to, pre-fetch logic to fetch instructions, decode logic to decode the instructions, execution logic to execute instructions and the like. In some aspects, processor 1910 has a cache memory 1916 to cache instructions and/or data for system 1900. Cache memory 1916 may be organized into a hierarchal structure including one or more levels of cache memory.

In some aspects, processor 1910 includes a memory controller 1914, which is operable to perform functions that enable the processor 1910 to access and communicate with memory 1930 that includes a volatile memory 1932 and/or a non-volatile memory 1934. In some aspects, processor 1910 is coupled with memory 1930 and chipset 1920. Processor 1910 may also be coupled to a wireless antenna 1978 to communicate with any device configured to transmit and/or receive wireless signals. In one aspect, an interface for wireless antenna 1978 operates in accordance with, but is not limited to, the IEEE 802.11 standard and its related family, Home Plug AV (HPAV), Ultra Wide Band (UWB), Bluetooth, WiMax, or any form of wireless communication protocol.

In some aspects, volatile memory 1932 includes, but is not limited to, Synchronous Dynamic Random Access Memory (SDRAM), Dynamic Random Access Memory (DRAM), RAMBUS Dynamic Random Access Memory (RDRAM), and/or any other type of random access memory device. Non-volatile memory 1934 includes, but is not limited to, flash memory, phase change memory (PCM), read-only memory (ROM), electrically erasable programmable read-only memory (EEPROM), or any other type of non-volatile memory device.

Memory 1930 stores information and instructions to be executed by processor 1910. In one aspect, memory 1930 may also store temporary variables or other intermediate information while processor 1910 is executing instructions. In the illustrated aspect, chipset 1920 connects with processor 1910 via Point-to-Point (PtP or P-P) interfaces 1917 and 1922. Chipset 1920 enables processor 1910 to connect to other elements in system 1900. In some aspects of the example system, interfaces 1917 and 1922 operate in accordance with a PtP communication protocol such as the Intel® QuickPath Interconnect (QPI) or the like. In other aspects, a different interconnect may be used.

In some aspects, chipset 1920 is operable to communicate with processor 1910, 1905, display device 1940, and other devices, including a bus bridge 1972, a smart TV 1976, I/O devices 1974, nonvolatile memory 1960, a storage medium (such as one or more mass storage devices) 1962, a keyboard/mouse 1964, a network interface 1966, and various forms of consumer electronics 1977 (such as a PDA, smart phone, tablet etc.), etc. In one aspect, chipset 1920 couples with these devices through an interface 1924. Chipset 1920 may also be coupled to a wireless antenna 1978 to communicate with any device configured to transmit and/or receive wireless signals.

Chipset 1920 connects to display device 1940 via interface 1926. Display 1940 may be, for example, a liquid crystal display (LCD), a plasma display, cathode ray tube (CRT) display, or any other form of visual display device. In some aspects of the example system, processor 1910 and chipset 1920 are merged into a single SOC. In addition, chipset 1920 connects to one or more buses 1950 and 1955 that interconnect various system elements, such as I/O devices 1974, nonvolatile memory 1960, storage medium 1962, a keyboard/mouse 1964, and network interface 1966. Buses 1950 and 1955 may be interconnected together via a bus bridge 1972.

In one aspect, mass storage device 1962 includes, but is not limited to, a solid-state drive, a hard disk drive, a universal serial bus flash memory drive, or any other form of computer data storage medium. In one aspect, network interface 1966 is implemented by any type of well-known network interface standard including, but not limited to, an Ethernet interface, a universal serial bus (USB) interface, a Peripheral Component Interconnect (PCI) Express interface, a wireless interface and/or any other suitable type of interface. In one aspect, the wireless interface operates in accordance with, but is not limited to, the IEEE 802.11 standard and its related family, Home Plug AV (HPAV), Ultra Wide Band (UWB), Bluetooth, WiMax, or any form of wireless communication protocol.

While the modules shown in FIG. 12 are depicted as separate blocks within the system 1900, the functions performed by some of these blocks may be integrated within a single semiconductor circuit or may be implemented using two or more separate integrated circuits. For example, although cache memory 1916 is depicted as a separate block within processor 1910, cache memory 1916 (or selected aspects of 1916) can be incorporated into processor core 1912.

Discussions herein utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

The terms “plurality” and “a plurality”, as used herein, include, for example, “multiple” or “two or more”. For example, “a plurality of items” includes two or more items.

References to “one aspect”, “an aspect”, “an example aspect”, “some aspects”, “demonstrative aspect”, “various aspects” etc., indicate that the aspect(s) so described may include a particular feature, structure, or characteristic, but not every aspect necessarily includes the particular feature, structure, or characteristic. Further, repeated use of the phrase “in one aspect” does not necessarily refer to the same aspect, although it may.

As used herein, unless otherwise specified the use of the ordinal adjectives “first”, “second”, “third” etc., to describe a common object, merely indicate that different instances of like objects are being referred to and are not intended to imply that the objects so described must be in a given sequence, either temporally, spatially, in ranking, or in any other manner.

Some aspects may be used in conjunction with various devices and systems, for example, a User Equipment (UE), a Mobile Device (MD), a wireless station (STA), a Personal Computer (PC), a desktop computer, a mobile computer, a laptop computer, a notebook computer, a tablet computer, a server computer, a handheld computer, a sensor device, an Internet of Things (IoT) device, a wearable device, a handheld device, a Personal Digital Assistant (PDA) device, a handheld PDA device, an on-board device, an off-board device, a hybrid device, a vehicular device, a non-vehicular device, a mobile or portable device, a consumer device, a non-mobile or non-portable device, a wireless communication station, a wireless communication device, a wireless Access Point (AP), a wired or wireless router, a wired or wireless modem, a video device, an audio device, an audio-video (A/V) device, a wired or wireless network, a wireless area network, a Wireless Video Area Network (WVAN), a Local Area Network (LAN), a Wireless LAN (WLAN), a Personal Area Network (PAN), a Wireless PAN (WPAN), and the like.

Some aspects may, for example, be used in conjunction with devices and/or networks operating in accordance with existing IEEE 802.11 standards (including IEEE 802.11-2016 (IEEE 802.11-2016, IEEE Standard for Information technology—Telecommunications and information exchange between systems Local and metropolitan area networks—Specific requirements Part 11: Wireless LAN Medium Access Control (MAC) and Physical Layer (PHY) Specifications, Dec. 7, 2016); IEEE 802.11ay (P802.11ay Standard for Information Technology—Telecommunications and Information Exchange Between Systems Local and Metropolitan Area Networks—Specific Requirements Part 11: Wireless LAN Medium Access Control (MAC) and Physical Layer (PHY) Specifications—Amendment: Enhanced Throughput for Operation in License-Exempt Bands Above 45 GHz)) and/or future versions and/or derivatives thereof, devices and/or networks operating in accordance with existing WiFi Alliance (WFA) Peer-to-Peer (P2P) specifications (including WiFi P2P technical specification, version 1.5, Aug. 4, 2015) and/or future versions and/or derivatives thereof, devices and/or networks operating in accordance with existing Wireless-Gigabit-Alliance (WGA) specifications (including Wireless Gigabit Alliance, Inc WiGig MAC and PHY Specification Version 1.1, April 2011, Final specification) and/or future versions and/or derivatives thereof, devices and/or networks operating in accordance with existing cellular specifications and/or protocols, e.g., 3rd Generation Partnership Project (3GPP), 3GPP Long Term Evolution (LTE) and/or future versions and/or derivatives thereof, units and/or devices which are part of the above networks, and the like.

Some aspects may be used in conjunction with one way and/or two-way radio communication systems, cellular radio-telephone communication systems, a mobile phone, a cellular telephone, a wireless telephone, a Personal Communication Systems (PCS) device, a PDA device which incorporates a wireless communication device, a mobile or portable Global Positioning System (GPS) device, a device which incorporates a GPS receiver or transceiver or chip, a device which incorporates an RFID element or chip, a Multiple Input Multiple Output (MIMO) transceiver or device, a Single Input Multiple Output (SIMO) transceiver or device, a Multiple Input Single Output (MISO) transceiver or device, a device having one or more internal antennas and/or external antennas, Digital Video Broadcast (DVB) devices or systems, multi-standard radio devices or systems, a wired or wireless handheld device, e.g., a Smartphone, a Wireless Application Protocol (WAP) device, or the like.

Some aspects may be used in conjunction with one or more types of wireless communication signals and/or systems, for example, Radio Frequency (RF), Infra-Red (IR), Frequency-Division Multiplexing (FDM), Orthogonal FDM (OFDM), Orthogonal Frequency-Division Multiple Access (OFDMA), Spatial Divisional Multiple Access (SDMA), FDM Time-Division Multiplexing (TDM), Time-Division Multiple Access (TDMA), Multi-User MIMO (MU-MIMO), Extended TDMA (E-TDMA), General Packet Radio Service (GPRS), extended GPRS, Code-Division Multiple Access (CDMA), Wideband CDMA (WCDMA), CDMA 2000, single-carrier CDMA, multi-carrier CDMA, Multi-Carrier Modulation (MDM), Discrete Multi-Tone (DMT), Bluetooth, Global Positioning System (GPS), Wi-Fi, Wi-Max, ZigBee™, Ultra-Wideband (UWB), Global System for Mobile communication (GSM), 2G, 2.5G, 3G, 3.5G, 4G, Fifth Generation (5G) mobile networks, 3GPP, Long Term Evolution (LTE), LTE advanced, Enhanced Data rates for GSM Evolution (EDGE), or the like. Other aspects may be used in various other devices, systems and/or networks.

The term “wireless device”, as used herein, includes, for example, a device capable of wireless communication, a communication device capable of wireless communication, a communication station capable of wireless communication, a portable or non-portable device capable of wireless communication, or the like. In some demonstrative aspects, a wireless device may be or may include a peripheral that is integrated with a computer, or a peripheral that is attached to a computer. In some demonstrative aspects, the term “wireless device” may optionally include a wireless service.

The term “communicating” as used herein with respect to a communication signal includes transmitting the communication signal and/or receiving the communication signal. For example, a communication unit, which is capable of communicating a communication signal, may include a transmitter to transmit the communication signal to at least one other communication unit, and/or a communication receiver to receive the communication signal from at least one other communication unit. The verb communicating may be used to refer to the action of transmitting and/or the action of receiving. In one example, the phrase “communicating a signal” may refer to the action of transmitting the signal by a first device and may not necessarily include the action of receiving the signal by a second device. In another example, the phrase “communicating a signal” may refer to the action of receiving the signal by a first device and may not necessarily include the action of transmitting the signal by a second device.

Some demonstrative aspects may be used in conjunction with a wireless communication network communicating over a frequency band above 45 Gigahertz (GHz), e.g., 60 GHz. However, other aspects may be implemented utilizing any other suitable wireless communication frequency bands, for example, an Extremely High Frequency (EHF) band (the millimeter wave (mmWave) frequency band), e.g., a frequency band within the frequency band of between 20 GHz and 300 GHz, a frequency band above 45 GHz, a frequency band below 20 GHz, e.g., a Sub 1 GHz (S1G) band, a 2.4 GHz band, a 5 GHz band, a WLAN frequency band, a WPAN frequency band, a frequency band according to the WGA specification, and the like.

As used herein, the term “circuitry” may, for example, refer to, be part of, or include, an Application Specific Integrated Circuit (ASIC), an integrated circuit, an electronic circuit, a processor (shared, dedicated, or group), and/or memory (shared, dedicated, or group), that execute one or more software or firmware programs, a combinational logic circuit, and/or other suitable hardware components that provide the described functionality. In some aspects, circuitry may include logic, at least partially operable in hardware. In some aspects, the circuitry may be implemented as part of and/or in the form of a radio virtual machine (RVM), for example, as part of a Radio processor (RP) configured to execute code to configured one or more operations and/or functionalities of one or more radio components.

The term “logic” may refer, for example, to computing logic embedded in circuitry of a computing apparatus and/or computing logic stored in a memory of a computing apparatus. For example, the logic may be accessible by a processor of the computing apparatus to execute the computing logic to perform computing functions and/or operations. In one example, logic may be embedded in various types of memory and/or firmware, e.g., silicon blocks of various chips and/or processors. Logic may be included in, and/or implemented as part of, various circuitry, e.g., radio circuitry, receiver circuitry, control circuitry, transmitter circuitry, transceiver circuitry, processor circuitry, and/or the like. In one example, logic may be embedded in volatile memory and/or non-volatile memory, including random access memory, read only memory, programmable memory, magnetic memory, flash memory, persistent memory, and/or the like. Logic may be executed by one or more processors using memory, e.g., registers, buffers, stacks, and the like, coupled to the one or more processors, e.g., as necessary to execute the logic.

The term “antenna” or “antenna array”, as used herein, may include any suitable configuration, structure and/or arrangement of one or more antenna elements, components, units, assemblies and/or arrays. In some aspects, the antenna may implement transmit and receive functionalities using separate transmit and receive antenna elements. In some aspects, the antenna may implement transmit and receive functionalities using common and/or integrated transmit/receive elements. The antenna may include, for example, a phased array antenna, a single element antenna, a set of switched beam antennas, and/or the like.

### Additional Notes and Aspects

Example 1 is an apparatus, comprising: an antenna; transmitter circuitry coupled to the antenna; and processing circuitry coupled to the transmitter circuitry and configured to: measure first reflection coefficient of the antenna in free space; measure a second reflection coefficient of the antenna when the antenna is proximate biological tissues; and determine a property of the biological tissues based on a comparison between the first reflection coefficient and second reflection coefficient.

In Example 2, the subject matter of Example 1 can optionally include wherein the property includes a measured substance concentration within the biological tissues or a physical characteristic of the biological tissues.

In Example 3, the subject matter of Example 2 can optionally include wherein the property includes blood glucose level.

In Example 4, the subject matter of any of Examples 1-3 can optionally include wherein the processing circuitry is configured to provide an indication to a user regarding a position of the antenna.

In Example 5, the subject matter of any of Examples 1˜4 can optionally include wherein the apparatus is configured to provide an indication regarding whether the biological tissues are correctly positioned relative to the antenna.

In Example 6, the subject matter of Example 5 can optionally include two or more antennas coupled to the processing circuitry, and wherein the processing circuitry is configured to measure reflection coefficients for each of the two or more antennas.

In Example 7, the subject matter of any of Examples 1-6 can optionally include wherein the antenna is configured to transmit a radio frequency signal.

In Example 8, the subject matter of any of Examples 1-7 can optionally include wherein the processing circuitry is configured to determine the property of the biological tissues based on an algorithm to establish a relationship between the property of biological tissues and a variation between the free space reflection coefficient and a body proximity reflection coefficient.

In Example 9, the subject matter of Example 8 can optionally include wherein the processing circuitry is configured to determine the property of the biological tissues based on a machine learning algorithm relating the property of biological tissues to a variation between the free space reflection coefficient and a body proximity reflection coefficient.

Example 10 is a device comprising: a user display; an antenna; transmitter circuitry coupled to the antenna; and processing circuitry coupled to the transmitter circuitry and configured to: measure a free space reflection coefficient of the antenna in free space; measure another reflection coefficient of the antenna when the antenna is proximate biological tissues; and determine a property of the biological tissues based on a comparison between the free space reflection coefficient and reflection coefficient proximate biological tissues.

In Example 11, the subject matter of Example 10 can optionally include wherein the processing circuitry is configured to provide an indication to a user regarding a position of the antenna.

In Example 12, the subject matter of Example 11 can optionally include wherein the processing circuitry is configured to: provide an indication regarding whether the biological tissues are correctly positioned relative to the antenna.

In Example 13, the subject matter of any of Examples 10-12 can optionally include wherein the antenna is configured to transmit a radio frequency signal.

In Example 14, the subject matter of Example 13 can optionally include two or more antennas coupled to the processing circuitry, and wherein the processing circuitry is configured to measure the reflection coefficients for each of the two or more antennas.

In Example 15, the subject matter of any of Examples 10-14 can optionally include wherein the device is a smart phone.

In Example 16, the subject matter of any of Examples 10-15 can optionally include wherein the device is a laptop computer.

Example 17 is a method comprising: providing an antenna; measuring a free space reflection coefficient of the antenna in free space; measuring another reflection coefficient of the antenna when the antenna is proximate biological tissues; and determining a property of the biological tissues based on a comparison between the free space reflection coefficient and reflection coefficient proximate biological tissues.

In Example 18, the subject matter of Example 17 can optionally include wherein the property includes a measured substance concentration within the biological tissues or a physical characteristic of the biological tissues.

In Example 19, the subject matter of any of Examples 17-18 can optionally include providing an indication to a user regarding a position of the antenna.

In Example 20, the subject matter of Example 19 can optionally include providing an indication regarding whether the biological tissues are correctly positioned relative to the antenna.

The above detailed description includes references to the accompanying drawings, which form a part of the detailed description. The drawings show, by way of illustration, specific aspects in which the invention can be practiced. These aspects are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other aspects can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed aspect. Thus, the following claims are hereby incorporated into the Detailed Description, with each claim standing on its own as a separate aspect, and it is contemplated that such aspects can be combined with each other in various combinations or permutations. The scope of the invention should be determined with reference to the appended claims, along with the full scope of equivalents to which such claims are legally entitled.

What is claimed is:

1. An apparatus comprising:
an antenna;
transceiver circuitry coupled to the antenna; and
processing circuitry coupled to the transceiver circuitry and configured to:
measure a first reflection coefficient of the antenna in free space;
measure a second reflection coefficient of the antenna when the antenna is proximate biological tissues; and
determine a contextual property of the biological tissues based on a comparison between the first reflection coefficient and second reflection coefficient.

2. The apparatus of claim 1, wherein the contextual property includes a measured substance concentration within the biological tissues or a physical characteristic of the biological tissues.

3. The apparatus of claim 2, wherein the contextual property includes blood glucose level.

4. The apparatus of claim 1, wherein the processing circuitry is configured to provide an indication to a user regarding a position of the antenna.

5. The apparatus of claim 1, wherein the apparatus is configured to provide an indication regarding whether the biological tissues are correctly positioned relative to the antenna.

6. The apparatus of claim 5, further comprising two or more antennas coupled to the processing circuitry, and wherein the processing circuitry is configured to measure reflection coefficients for each of the two or more antennas.

7. The apparatus of claim 1, wherein the antenna is configured to transmit a radio frequency signal.

8. The apparatus of claim 1, wherein the processing circuitry is configured to determine the contextual property of the biological tissues based on an algorithm to establish a relationship between the contextual property of biological tissues and a variation between the first reflection coefficient and a body proximity reflection coefficient.

9. The apparatus of claim 8, wherein the processing circuitry is configured to determine the contextual property of the biological tissues based on a machine learning algorithm relating the contextual property of biological tissues to a variation between the first reflection coefficient and a body proximity reflection coefficient.

10. A device comprising:
a display;
an antenna;
transmitter circuitry coupled to the antenna; and
processing circuitry coupled to the transmitter circuitry and configured to:
measure a free space reflection coefficient of the antenna in free space;
measure another reflection coefficient of the antenna when the antenna is proximate biological tissues; and
determine a contextual property of the biological tissues based on a comparison between the free space reflection coefficient and reflection coefficient proximate biological tissues.

11. The device of claim 10, wherein the processing circuitry is configured to provide an indication to a user regarding a position of the antenna.

12. The device of claim 11, wherein the processing circuitry is configured to:
provide an indication regarding whether the biological tissues are correctly positioned relative to the antenna.

13. The device of claim 10, wherein the antenna is configured to transmit a radio frequency signal.

14. The device of claim 13, further comprising two or more antennas coupled to the processing circuitry, and wherein the processing circuitry is configured to measure the reflection coefficients for each of the two or more antennas.

15. The device of claim 10, wherein the device is a smart phone.

16. The device of claim 10, wherein the device is a laptop computer.

17. A method comprising:
providing an antenna;
measuring a free space reflection coefficient of the antenna in free space;
measuring another reflection coefficient of the antenna when the antenna is proximate biological tissues; and
determining a contextual property of the biological tissues based on a comparison between the free space reflection coefficient and reflection coefficient proximate biological tissues.

18. The method of claim 17, wherein the contextual property includes a measured substance concentration within the biological tissues or a physical characteristic of the biological tissues.

19. The method of claim 17, further comprising providing an indication to a user regarding a position of the antenna.

20. The method of claim 19, further comprising:
providing an indication regarding whether the biological tissues are correctly positioned relative to the antenna.
